(19) United States
(12) Patent Application Publication
(10) Pub. No.: US 2012/0082735 A1
(43) Pub. Date: Apr. 5, 2012

(54) Allergic skin desease topical cream

(76) Inventor: Lyndon Mansfield, El Paso, TX (US)

(21) Appl. No.: 12/931,968

(22) Filed: Feb. 15, 2011

Related U.S. Application Data
(60) Provisional application No. 61/388,244, filed on Sep. 30, 2010.

(51) Int. Cl. A61K 33/30; A61P 37/08; A61P 17/00

(52) U.S. Cl. 424/642

(57) ABSTRACT

Disclosed is a cream formulation containing calamine, an H1 antihistamine, and a corticosteroid for the reduction of inflammation, redness and itching associated with acute and chronic allergic skin disease.

This application claims the benefit of the filing date, Feb. 16, 2010, of provisional application No. 61/388,244.

### BACKGROUND OF THE INVENTION

1. Field of the Invention

This invention relates to a topical cream used to inhibit both early and late phases of an allergic reaction in both acute and chronic allergic skin disease as well as a method of using same.

2. Description of the Prior Art

Allergy is the term given to a reaction by a small number of people to a substance, known as an allergen, which is harmless to those who are not allergic to it. Allergic contact dermatitis is inflammation of the skin manifested by varying degrees of erythema (redness of the skin), edema (swelling) and vesiculation (blistering). It is a delayed type of induced sensitivity (allergy) resulting from cutaneous contact with a specific allergen to which a person has developed a sensitivity. Allergic contact dermatitis becomes complicated by bacterial and fungal infestation or infection from scratching of the infected area. Understanding the benefits of the topical cream of the present invention requires a basic understanding of what causes the allergic reaction.

A properly functioning immune system is responsible for, among other things, the identification and destruction of cells which are infected with viruses. Allergic reactions are the result of an immune system that is not functioning properly but which misidentifies an otherwise innocuous substance as harmful. Lymphocytes, a type of white blood cell, are a fundamental component of the body's immune system. Lymphocytes move freely through and within the tissues of the body, travel through the walls of blood vessels and move between the various lymph nodes. When a lymphocyte encounters a foreign substance, the allergen, it will move its way back to a lymph node, change into a plasma cell and produce antibodies specifically engineered to attack the allergen.

There are five basic types of antibodies, called immunoglobulins or “Igs.” The Ig responsible for allergic reactions is IgE. The binding of an allergen to a specific IgE located on a mast cell or basophil causes the release of granules from these cells. Basophils and mast cells are types of cells that contain histamine and other allergy mediators. The released granules contain the histamine and other allergy mediators which causes allergic inflammation in 2 phases, an early immediate phase and a delayed onset (2 hours or more) late phases. Histamine acts on the small blood cells making them dilate causing redness (erythema) and swelling (edema). Histamine also stimulates nerve endings causing pain and itching. Histamine acts to recruit other inflammatory cells into the tissue. Sensitized lymphocytes promote mast cell activation and growth. Exogenous bacteria or fungus are introduced when the reactions are scratched and the epithelial barrier is penetrated inducing additional inflammatory responses.

Atopic Dermatitis is a rashing disease which resembles contact dermatitis but generally occurs in younger children, has a widespread distribution, and is triggered by ingested food allergens, airborne allergens and normal skin bacteria. LgE allergy appears to be even more dominant in these types of skin rash reaction which like contact dermatitis also has an early and late phase component.

### SUMMARY OF THE INVENTION

It is, therefore, an object of the present invention to provide a topical cream which inhibits early as well as late states of an allergic reaction in the treatment of acute and chronic allergic contact as well as atopic dermatitis. It is also an object of this invention to provide for a method of use of the topical cream. Other objects and advantages of the present invention will become apparent from the following detailed description which set forth certain embodiments of the invention.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as the basis for the claims and as a basis for teaching one skilled in the art how to make and/or use the invention.

Calamine, which is a combination of zinc oxide with iron oxide, is an astringent and antibacterial as well as antifungal. Antihistamines block the histamine reaction reducing swelling and itching. Corticosteroids are anti-inflammatory and interfere with the flow of cells to the infected site caused by the recruitment by allergy mediators. A cream containing the combination of a calamine with an H1 Antihistamine as well as a corticosteroid will therefore fight infection and reduce both swelling and itching. In practice, any combination of calamine, a corticosteroid and H1 antihistamine will be effective however in the present invention an approximate ratio of 6 grams of 0.1% triamicinolone cream (a corticosteroid) is used per 1 ml of calamine lotion. To this mixture is added 1 mg of diphenhydramine (an antihistamine). The resulting cream is then applied to the allergy affected site.

1. A topical cream comprising calamine, an H1 antihistamine and a corticosteroid.

2. The method of using the cream of claim 1 comprising the steps of applying said cream to the allergy affected site.
